(19) United States
(12) Patent Application Publication
(10) Pub. No.: US 2026/0021235 A1
(43) Pub. Date: Jan. 22, 2026

(54) SYSTEMS AND METHODS FOR BLOOD RETURN IN A HEMODIALYSIS MACHINE

(71) Applicants: Fresenius Medical Care Deutschland Gmbh, Bad Homburg (DE); Fresenius Medical Care Holdings, Inc., Waltham, MA (US)

(72) Inventors: Christian Schlaeper, Wehrheim (DE); David Yuds, Hudson, NH (US); Martin Crnkovich, Walnut Creek, CA (US)

(73) Assignees: Fresenius Medical Care Deutschland Gmbh, Bad Homburg (DE); Fresenius Medical Care Holdings, Inc., Waltham, MA (US)

(21) Appl. No.: 18/778,019

(22) Filed: Jul. 19, 2024

(51) Int. Cl. A61M 1/36; A61M 39/22; A61M 60/113; A61M 60/37

(52) U.S. Cl. CPC A61M 1/3661; A61M 39/223; A61M 60/113; A61M 60/37; A61M 2039/226

(57) ABSTRACT

The present disclosure describes systems and methods for blood return in a hemodialysis machine using an adjustable valve that includes a first portion having a first port configured to be connected to a machine arterial line and a second port configured to be connected to a machine venous line, and a second portion of the adjustable valve having an arterial inlet port configured to be connected to a patient arterial access line, a venous outlet port configured to be connected to a patient venous access line, and a substitution inlet port configured to be connected to a substitution fluid line. The first portion can be movable relative to the second portion to selectively connect the first port and the second port with a selected combination of two of the arterial inlet port, the venous outlet port, or the substitution inlet port to adjust a flow of fluid therethrough.

### TECHNICAL FIELD

The present disclosure relates generally to dialysis systems and methods. More particularly, the present disclosure relates to systems and methods for clearing blood from an arterial bloodline of a hemodialysis machine.

### BACKGROUND

Many modern medical procedures use tubing sets of varying complexity to withdraw fluid from a patient, or to administer fluid to a patient, or to do both. One example of such a procedure is hemodialysis. In hemodialysis, the patient's blood is cleansed by drawing it out of the patient through a blood access site, typically via a catheter, and passing it through an artificial kidney (often called a “dialyzer”). The artificial kidney includes a semi-permeable membrane which removes impurities and toxins by a process of diffusion. The purified blood is then returned to the patient. An extracorporeal circuit including a pump and hemodialysis tubing set is typically used to transport the blood between the blood access site and the artificial kidney.

At the end of a hemodialysis session, the patient's blood remaining in the machine blood circuit is rinsed back using saline from a saline bag or substitution fluid from the machine's online hemodiafiltration (HDF) system. In some situations, the blood in the patient's arterial access line is either discarded or manually reinfused back into the patient using a saline-and/or substitution-fluid-filled syringe or bag. Even small amounts of blood discarded from the arterial access line may accumulate to a meaningful amount over time, however, which can require the supplementation of additional erythropoietin. Thus, it is preferred that this blood should also be rinsed back to the patient.

In some advanced machines, the arterial blood pump can be run backwards at the end of treatment to automatically return arterial blood through the patient's arterial access, using an air bubble detector to monitor the process. Many systems do not have an air bubble detector on the arterial line, however, and are thus not configured to safely run in reverse. For such systems, the blood in the patient's arterial access line up to the saline bag/substitution fluid port connection must be manually reinfused. Conventionally, squeezing a saline bag is imprecise and done with no pressure monitoring on the access, so to perform this reinfusion, the clinician must perform a sequence of steps, which can include (1) filling a new, sterile syringe with saline; (2) clamping both the machine arterial line and the patient arterial access line; (3) disconnecting the patient arterial access line; (4) connecting the patient arterial access line to the end of the machine's arterial line; and then (5) carefully reinfusing the blood back to the patient. In addition, manually filling a syringe with saline is time-consuming, and using pre-filled saline syringes is more costly. In either case, the syringe must be discarded after the procedure, adding materials and labor costs to every treatment.

It is with respect to these and other considerations that the present disclosure may be useful.

### SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

In accordance with this disclosure, systems, and methods for blood return in a hemodialysis machine are provided. In some embodiments, a system for blood return includes an adjustable valve comprising: a first portion having a first port configured to be connected to a machine arterial line and a second port configured to be connected to a machine venous line; and a second portion having an arterial inlet port configured to be connected to a patient arterial access line, a venous outlet port configured to be connected to a patient venous access line, and a substitution inlet port configured to be connected to a substitution fluid line. The first portion of the adjustable valve can be movable relative to the second portion to selectively connect the first port and the second port with a selected combination of two of the arterial inlet port, the venous outlet port, or the substitution inlet port to adjust a flow of fluid through the adjustable valve.

In another embodiment, a dialysis machine is provided in which an adjustable valve has a first portion having a first port and a second port and a second portion having an arterial inlet port configured to be connected to a patient arterial access line, a venous outlet port configured to be connected to a patient venous access line, and a substitution inlet port. The dialysis machine further includes a machine arterial line comprising a first end and a second end substantially opposing the first end, where the first end is connected to the first port, and where the second end is connected to an input port of a dialyzer; an arterial blood pump in communication with the machine arterial line and configured to pump fluid from the first end to the second end; a machine venous line comprising a first end and a second end substantially opposing the first end, where the first end is connected to an output port of the dialyzer, and where the second end is connected to the second port; and a substitution fluid line comprising a first end and a second end substantially opposing the first end, where the first end is connected to a substitution fluid source, and where the second end is connected to the substitution inlet port. In this configuration, the first portion of the adjustable valve is movable relative to the second portion to selectively connect the first port and the second port with a selected combination of two of the arterial inlet port, the venous outlet port, or the substitution inlet port to adjust a flow of fluid through the adjustable valve.

In a further embodiment, a method for blood return in a hemodialysis machine is provided. The method can use an adjustable valve comprising a first portion having a first port connected to a machine arterial line and a second port connected to a machine veinous line and a second portion having an arterial inlet port connected to a patient arterial access line, a venous outlet port connected to a patient venous access line, and a substitution inlet port connected to a substitution fluid line. In this way, the method can involve moving the first portion of the adjustable valve to a first position relative to the second portion in which the first port and the second port are connected with a first combination of two of the arterial inlet port, the venous outlet port, or the substitution inlet port; operating an arterial blood pump in communication with the machine arterial line to control a first flow of fluid through the adjustable valve to the machine arterial line; moving the first portion to a second position relative to the second portion in which the first port and the second port are connected with a second combination of two of the arterial inlet port, the venous outlet port, or the substitution inlet port; and operating the arterial blood pump to control a second flow of fluid through the adjustable valve to the machine arterial line.

In any preceding or subsequent example, the first portion can be rotatably coupled to the second portion.

In any preceding or subsequent example, the adjustable valve can be movable to one or more discrete alignment positions. In some examples, these discrete alignment positions can include two or more of a first position in which the first port is in communication with the arterial inlet port and the second port is in communication with the venous outlet port; a second position in which the first port is in communication with the substitution inlet port and the second port is in communication with the venous outlet port; a third position in which the first port is connected with the substitution inlet port and the second port is connected with the arterial inlet port; and/or a fourth position in which the first port is connected with the venous outlet port and the second port is connected with the arterial inlet port.

In any preceding or subsequent example, a coupling interface between the first portion and the second portion can include one or more detent configured to provide physical feedback to identify alignment of the first portion and the second portion in one or more alignment positions.

In any preceding or subsequent example, the first portion can include a sealing element configured to block flow through the one of the arterial inlet port, the venous outlet port, or the substitution inlet port that is not connected to either of the first port or the second port.

In any preceding or subsequent example, a control system can be provided in communication with the arterial blood pump and configured to control the flow of fluid through the adjustable valve to the machine arterial line.

In any preceding or subsequent example, an actuator can be connected to the adjustable valve and configured to adjust a position of the first portion relative to the second portion in response to a control signal.

An adjustable valve of this kind can advantageously change the fluid flow through fluid lines connected thereto without requiring repositioning, twisting, and/or reconnection of the fluid lines relative to one another. Reduced repositioning or twisting of the fluid lines can result in less kinking or binding of the fluid lines and, as a result, better flow through the fluid lines. This configuration may also reduce the risk of cross contamination and blood loss due to loose connections.

Although some of the features of the present disclosure have been stated hereinabove, and which are achieved in whole or in part, other feature will become evident as the description proceeds when taken in connection with the accompanying drawings as best described hereinbelow.

### BRIEF DESCRIPTION OF THE DRAWINGS

The features and advantages of the present disclosure will be more readily understood from the following detailed description which should be read in conjunction with the accompanying drawings that are given merely by way of explanatory and non-limiting example, and in which:

FIG. 1 is a schematic plan view of a dialysis machine blood circuit including a blood return system according to an embodiment of the present disclosure.

FIG. 2A is a side view of components of an adjustable valve for use with the blood return system according to an embodiment of the present disclosure.

FIG. 2B is a top view of an adjustable valve for use with the blood return system according to an embodiment of the present disclosure.

FIG. 3 is a plan view of a dialysis machine blood circuit including a blood return system according to an embodiment of the present disclosure.

FIGS. 4A-4D are schematic top views of an adjustable valve in various flow positions and associated plan views of the blood return system according to an embodiment of the present disclosure.

It should be understood that the drawings are not necessarily to scale and that the disclosed embodiments are sometimes illustrated diagrammatically and in partial views. In certain instances, details which are not necessary for an understanding of the disclosed methods and devices or which render other details difficult to perceive may have been omitted. It should be further understood that this disclosure is not limited to the particular embodiments illustrated herein. In the drawings, like numbers refer to like elements throughout unless otherwise noted.

### DETAILED DESCRIPTION

The present disclosure provides systems and methods for blood return in a hemodialysis machine. Various features or the like of a blood return system will now be described more fully herein with reference to the accompanying drawings, in which one or more features of the blood return system will be shown and described. It should be appreciated that the various features may be used independently of, or in combination, with each other. It will be appreciated that the blood return system as disclosed herein may be embodied in many different forms and may selectively include one or more concepts, features, or functions described herein. As such, the blood return system should not be construed as being limited to the specific examples set forth herein. Rather, these examples are provided so that this disclosure will convey certain features of the blood return system to those skilled in the art.

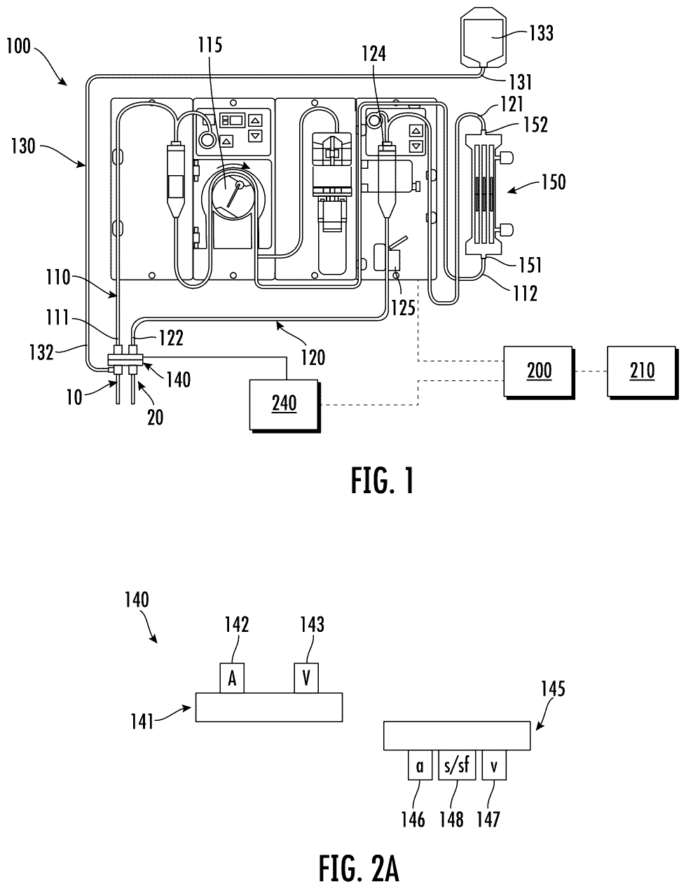

In accordance with one or more features of the present disclosure, the present disclosure provides systems and methods for using the blood flow reversal action of an adjustable three-way valve connected to the arterial and venous bloodlines during a blood return process. Referring to an example embodiment shown in FIG. 1, normal operation of a dialysis machine blood circuit, generally designated 100, can include a machine arterial line 110 having a first end 111 that is configured to be connected to a patient arterial access line 10 by an adjustable valve 140. In some embodiments, an arterial blood pump 115 connected to the machine arterial line 110 is configured to pump the blood to a second end 112 of the machine arterial line 110 substantially opposing the first end 111, which is configured to be connected to an input port 151 of a dialyzer, generally designated 150. Blood supplied to the dialyzer 150 can thereby be cleaned or filtered through diffusion and convection exchanges between the blood and a dialysate that is simultaneously pumped through the dialyzer 150. Operation of one or more components of the machine blood circuit 100 can be controlled by a control system 200, and a user interface 210 can be connected to the control system 200. In this way, operation of the machine blood circuit 100 can be easily managed by the operator and/or the operator can be prompted to perform various steps in the operation of the machine blood circuit 100.

After processing by the dialyzer 150, the blood can then be transported from an output port 152 of the dialyzer 150 to a first end 121 of a machine venous line 120 connected thereto. A second end 122 of the machine venous line 120 substantially opposing the first end 121 can then further be connected to a patient venous access line 20 by the adjustable valve 140 to return the processed blood to the patient. In some embodiments, the machine venous line 120 includes a venous drip chamber 124 that is configured to regulate blood flow and/or an air bubble detector 125 that is configured to detect entrained air within the blood flow at a designed position between the first end 121 and the second end 122 of the machine venous line 120.

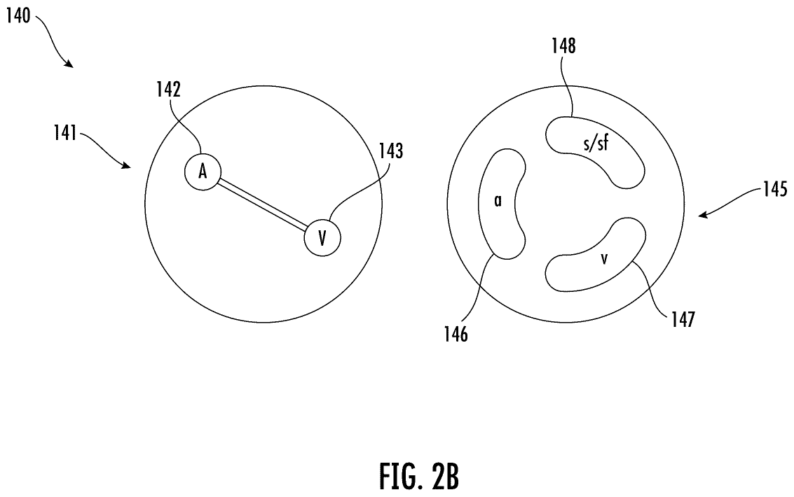

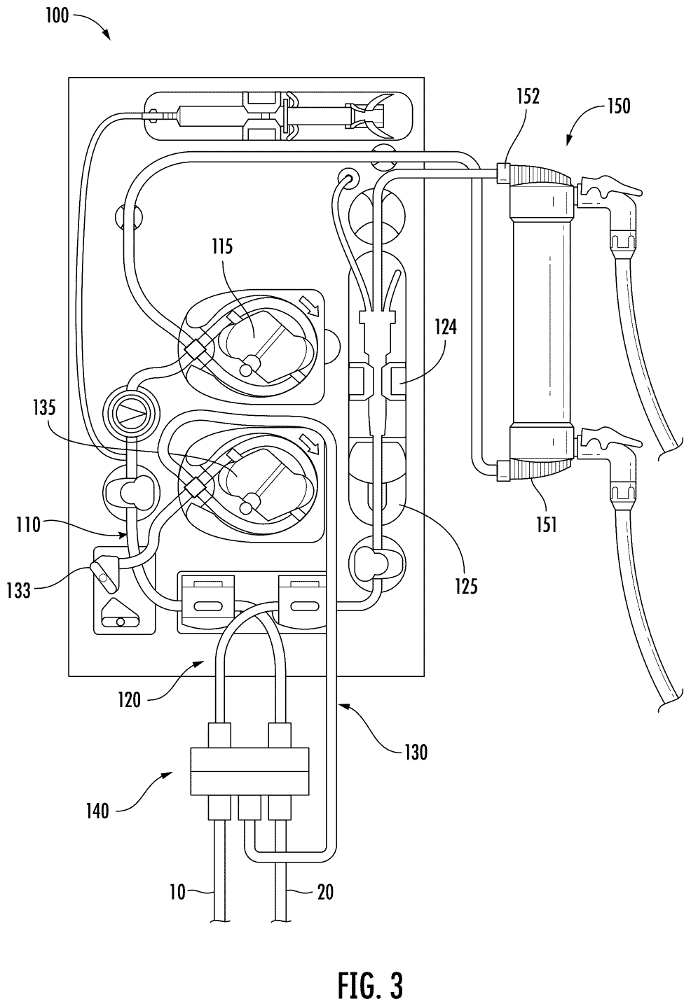

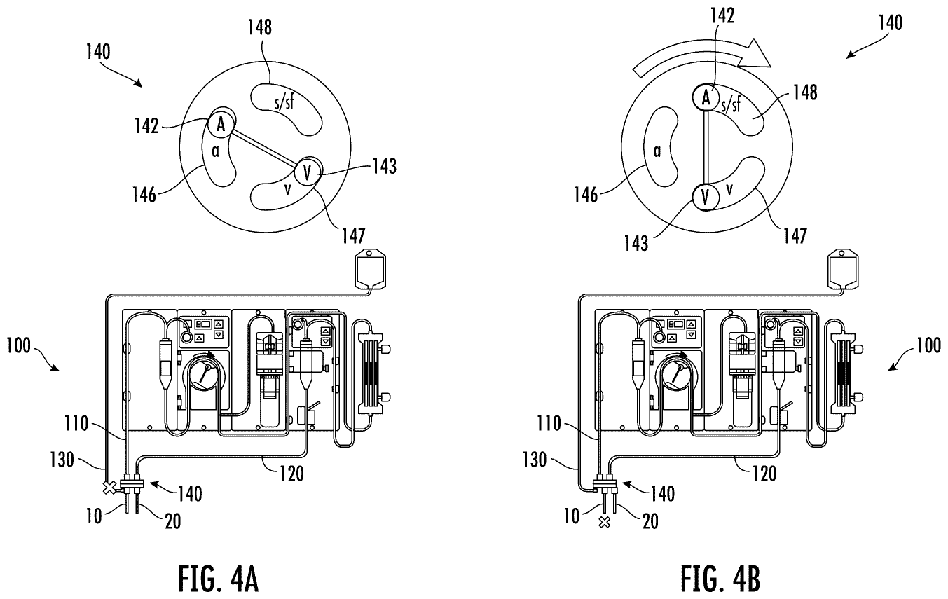

As indicated above, for normal operation of the dialysis machine blood circuit 100, the adjustable valve 140 can be arranged in a first position in which the machine arterial line 110 is connected to the patient arterial access line 10 (i.e., arterial-arterial connection) and the machine venous line 120 is connected to the patient venous access line 20 (i.e., a venous-venous position of the adjustable valve 140). Referring to FIGS. 2A and 2B, in some embodiments, this arrangement is achieved by the adjustable valve 140 having a first portion 141 that includes a first port 142 that is configured to be connected to the machine arterial line 110 and a second port 143 that is configured to be connected to the machine venous line 120. The adjustable valve 140 further includes a second portion 145 having an arterial inlet port 146 that is configured to be connected to the patient arterial access line 10, a venous outlet port 147 that is configured to be connected to the patient venous access line 20, and a substitution inlet port 148 that is configured to be connected to a substitution fluid line 130. In some embodiments, the substitution fluid line 130 includes a first end 131 that is connected to a substitution fluid source 133 and a second end 132 substantially opposing the first end 131 that is connected to the adjustable valve 140. In some embodiments, the substitution fluid source 133 can be a saline bag or other external fluid source. Alternatively, in some embodiments, the substitution fluid source 133 is a source of substitution fluid that is generated onboard the machine, such as is shown in FIG. 3. In such an embodiment, the machine blood circuit 100 can further include a substitution fluid pump 135 that is configured to control a flow of fluid through the substitution fluid line 130. In any configuration, each of the ports of the adjustable valve 140 is configured to be fluidically connected to a corresponding fluid line, such as by sliding the end of each fluid line over the respective port and securing (e.g., adhesively attaching) the end of the line to the port.

The first portion 141 and the second portion 145 are configured to be selectively coupled together in any of a variety of positions to selectively control the inputs and outputs to the machine blood circuit 100. In some embodiments, for example, the first portion 141 includes a generally cylindrical first valve body, the second portion 145 includes a generally cylindrical second valve body, and one or more central rotatable flow directing element (not shown) is disposed in a cavity formed between the first and second valve bodies. In some embodiments, flow directing elements are provided in each of the first portion 141 and the second portion 142 to direct both of the flows out of one portion of the adjustable valve 140 and into the other portion of the adjustable valve 140.

Alternatively, in some embodiments, the adjustable valve 140 is a rotatably-connected flow reversal valve similar to the device disclosed in U.S. Pat. No. 9,415,151, except that the adjustable valve 140 is adapted to have a third port in the second portion 142 that is configured to be coupled to the substitution fluid line 130.

In addition, the elements of the adjustable valve 140 can be sized and/or configured to create a fluid-tight coupling between the first portion 141 and the second portion 145, such as by including one or more gaskets or other sealing mechanisms. In some embodiments, the first portion 141 and the second portion 145 are formed of generally disk-shaped end plates and circumferentially formed cylindrical walls that extend from the end plates. In some embodiments, the first portion 141 and the second portion 145 are axially fixed but rotatably movable relative to one another such that the flow directing element is rotatable within the cavity formed therebetween. Alternatively, in some embodiments, the first portion 141 and the second portion 145 are axially and rotationally fixed relative to one another, and the flow directing element is rotatable within the cavity formed therebetween to change the position of flow channels within the adjustable valve 140 to connect a desired set of ports for fluid communication together.

In any configuration, because of the mismatch between the number of ports of the first portion 141 and the number of ports of the second portion 145, the first portion 141 can include a sealing element configured to block flow through the one of the arterial inlet port 146, the venous outlet port 147, or the substitution inlet port 148 that is not connected to either of the first port 142 or the second port 143. In some embodiments, for example, both the tubing and the sealing surface can be designed to have a low material hardness such that compression of the elements can be pressed together to create a firm seal when in a desired alignment position. In addition, in such configurations, the warmth of the blood and saline/substitution fluid can act to further soften the material and allow for a tighter seal.

The first portion 141 and the second portion 145 (and any internal components of the adjustable valve 140) can be made of one or more biocompatible high-impact thermoplastic or thermoset materials. In some embodiments, for example, the elements of the adjustable valve 140 are formed of acrylic-based multipolymer compound (e.g., a biocompatible high impact MMA/styrene/acrylonitrile terpolymer or similar injection moldable thermoplastic compound). Those having ordinary skill in the art will recognize, however, that other medical grade materials, such as polycarbonate, polysulfone, or blends of these types of materials, can alternatively or additionally be used. The elements of the adjustable valve 140 can be formed using any of a variety of known techniques, including but not limited to injection molding, etching, and/or machining.

In any configuration, the alignment of the first portion 141 and the second portion 145 is adjustable by moving (e.g., rotating) the first portion 141 with respect to the second portion 145 such that the first port 142 and the second port 143 are fluidically connected to a selected combination of two of the arterial inlet port 146, the venous outlet port 147, or the substitution inlet port 148. In some embodiments, the coupling interface between the two portions can further include one or more detent and/or step in the bezel, which can be arranged to provide physical/haptic and/or auditory feedback to the user to identify that the first portion 141 and the second portion 145 are correctly arranged in one or more alignment positions. In some embodiments, such a detent can include one or more snap-in style resilient fingers that deflect and lock in place to connect the first portion 141 and the second portion 145. Alternatively or in addition, identification of the arrangement of the first portion 141 and the second portion 145 in one of the alignment positions can be provided using a color coding scheme or line marks that identify the relative positions of the portions. In this way, the first portion 141 and the second portion 145 can be moved relative to one another to any of a variety of discrete positions that correspond to different fluid flow patterns. Further, when the first portion 141 and the second portion 145 are properly arranged in each of these alignment positions, fluid flow can be provided among the desired flow paths while sealing off the unconnected channel.

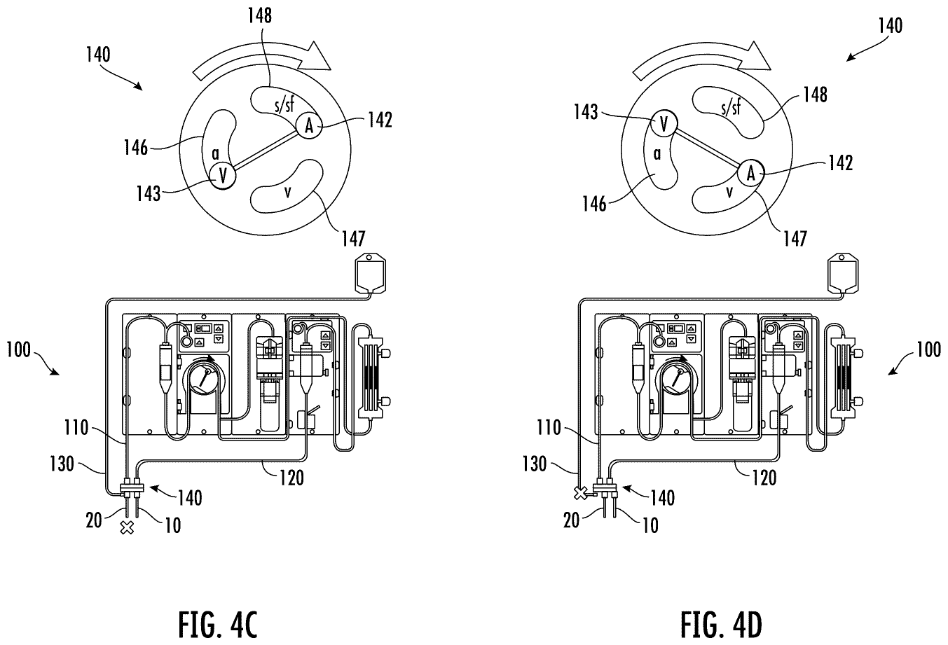

For example, as discussed above, normal operation can be achieved by arranging the adjustable valve 140 in a first position in which the machine arterial line 110 is in fluid communication with the patient arterial access line 10, and the machine venous line 120 is in fluid communication with the patient venous access line 20. As shown in FIG. 4A, this first position can be achieved by positioning the portions of the adjustable valve 140 to connect the first port 142 with the arterial inlet port 146 and connect the second port 143 with the venous outlet port 147, leaving the substitution inlet port 148 unconnected to either port of the first portion 141.

When the treatment has been completed, common practice involves running saline or substitution fluid through the machine arterial line 110 and the rest of the extracorporeal circuit (i.e., through the dialyzer 150 and the machine venous line 120) to return almost all the blood to the patient via the patient venous access line 20. To enable this configuration, the adjustable valve 140 can be moved to a second position in which the first portion 141 is moved (e.g., rotated) relative to the second portion 145 such that the first port 142 is connected with the substitution inlet port 148, the second port 143 is connected with the venous outlet port 147, and the arterial inlet port 146 is left unconnected to either port of the first portion 141. An example of the adjustable valve 140 being arranged in this second position is shown in FIG. 4B. In this arrangement, no further blood is drawn from the patient via the patient arterial access line 10. Instead, fluid is drawn from the substitution fluid source 133 through the substitution fluid line 130 (e.g., by further operation of the arterial blood pump 115) to be flowed through the machine arterial line 110 and the rest of the extracorporeal circuit (i.e., through the dialyzer 150 and the machine venous line 120), effectively rinsing the entire extracorporeal circuit of blood.

Upon completion of this primary rinse, however, the blood in the patient's arterial access line 10 may still remain. In some embodiments, the adjustable valve 140 can further be configured to be moved to a third position, such as is shown in FIG. 4C, in which the first portion 141 is moved relative to the second portion 145 such that the first port 142 is remains connected with the substitution inlet port 148, but the second port 143 is connected with the arterial inlet port 146, and the venous outlet port 147 is left unconnected to either port of the first portion 141. In this position, the blood in the patient's arterial access line 10 is now linked to the machine venous line 120, and the hemodialysis machine may run the arterial blood pump 115 another rotation or two to automatically return the last of the patient's blood back through the arterial access line 10. In this arrangement, the existing venous air bubble detector 125 can be used to ensure safe return of the blood, and this method also has the advantage of pressure monitoring using the existing safety system.

Finally, in some embodiments, the adjustable valve 140 can further be configured to be moved to a fourth position, such as is shown in FIG. 4D, in which the first port 142 is connected with the venous outlet port 147, the second port 143 is connected with the arterial inlet port 146, and the substitution inlet port 148 is left unconnected to either port of the first portion 141. This alignment in which the arterial and venous lines are effectively reversed can be used, for example, for situations like priming, saline boluses during treatment, automated access flow measurements, and the above-described rinse back process.

Using the adjustable valve 140 as discussed above, the requirement to selectively clamp the patient arterial access line 10, the patient venous access line 20, the machine arterial line 110, the machine venous line 120, and/or the substitution fluid line 130 at certain times to control the flows into and out of the system is removed. The incorporation of the adjustable valve 140 thus both simplifies the operation of the system 100 and eliminates the need for clamping devices for each dedicated line, which can provide further cost savings.

As discussed above, operation of one or more components of the machine blood circuit 100 can be controlled by a control system 200, and a user interface 210 can be connected to the control system 200. In some embodiments, for example, on-screen prompts displayed on the user interface 210 can guide the operator through the entire process to change the configuration of the adjustable valve 140 at the appropriate times. Alternatively or in addition, the operation of the adjustable valve 140 can be configured to be controlled directly by the control system 200 so the entire blood return process is fully automatic. In some embodiments, for example, the control system 200 can be in communication with an actuator 240 connected to the adjustable valve 140 (See, e.g., FIG.

1). Control signals can be sent from the control system 200 to the actuator 240 to operate the adjustable valve 140. In some embodiments, the control system 200 is programmed to transmit control signals to rotate the adjustable valve 140 and thus change the fluid flow through the adjustable valve 140 at designated times during treatment. In such implementations, the control system 200 can receive signals from a timer indicating how long the treatment has been underway and can cause the adjustable valve 140 to rotate when a predetermined time is reached. Alternatively, the control system 200 can be configured to transmit signals to the actuator 240 to change the position of the adjustable valve 140 based on operator input via the user interface 210.

Although the present disclosure is discussed in relation to operation of a clinical hemodialysis machine, the principles discussed herein can similarly be used in home hemodialysis machines to easily add functionality like access flow tests and automatic saline bolus. The use of the adjustable valve 140 could also be adapted to make peritoneal dialysis (PD) much simpler, compact, and lighter with a one direction pump or automated valve. In such implementations, the automated peritoneal dialysis (APD) cycler or continuous ambulatory peritoneal dialysis (CAPD) cycler could be configured to simply rotate the adjustable valve 140 to change flow directions from the fresh dialysate bag to the patient to the drain.

The present disclosure can be embodied in other forms without departure from the spirit and essential characteristics thereof. The embodiments described therefore are to be considered in all respects as illustrative and not restrictive. Although the present disclosure has been described in terms of certain preferred embodiments, other embodiments that are apparent to those of ordinary skill in the art are also within the scope of the present disclosure.

As used herein, an element or operation recited in the singular and proceeded with the word “a” or “an” should be understood as not excluding plural elements or operations, unless such exclusion is explicitly recited. Furthermore, references to “one embodiment” or “one example” of the present disclosure are not intended to be interpreted as excluding the existence of additional embodiments or examples that also incorporate the recited features.

The present disclosure is not to be limited in scope by the specific embodiments or examples described herein. Indeed, other various embodiments or examples and modifications to the present disclosure, in addition to those described herein, will be apparent to those of ordinary skill in the art from the foregoing description and accompanying drawings. Thus, such other embodiments or examples and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

What is claimed is:

1. A system for blood return in a hemodialysis machine, the system comprising:
an adjustable valve comprising:
a first portion having a first port configured to be connected to a machine arterial line and a second port configured to be connected to a machine venous line; and
a second portion having an arterial inlet port configured to be connected to a patient arterial access line, a venous outlet port configured to be connected to a patient venous access line, and a substitution inlet port configured to be connected to a substitution fluid line;
wherein the first portion of the adjustable valve is movable relative to the second portion to selectively connect the first port and the second port with a selected combination of two of the arterial inlet port, the venous outlet port, or the substitution inlet port to adjust a flow of fluid through the adjustable valve.

2. The system of claim 1, wherein the first portion is rotatably coupled to the second portion.

3. The system of claim 1, wherein the adjustable valve is movable to one or more discrete alignment positions including two or more of:
a first position in which the first port is in communication with the arterial inlet port and the second port is in communication with the venous outlet port;
a second position in which the first port is in communication with the substitution inlet port and the second port is in communication with the venous outlet port;
a third position in which the first port is connected with the substitution inlet port and the second port is connected with the arterial inlet port; and
a fourth position in which the first port is connected with the venous outlet port and the second port is connected with the arterial inlet port.

4. The system of claim 3, wherein a coupling interface between the first portion and the second portion comprises one or more detent configured to provide physical feedback to identify alignment of the first portion and the second portion in one or more alignment positions.

5. The system of claim 1, wherein the first portion comprises a sealing element configured to block flow through the one of the arterial inlet port, the venous outlet port, or the substitution inlet port that is not connected to either of the first port or the second port.

6. The system of claim 1, comprising an actuator connected to the adjustable valve and configured to adjust a position of the first portion relative to the second portion in response to a control signal.

7. A dialysis machine comprising:
an adjustable valve comprising:
a first portion having a first port and a second port; and
a second portion having an arterial inlet port configured to be connected to a patient arterial access line, a venous outlet port configured to be connected to a patient venous access line, and a substitution inlet port;
a machine arterial line comprising a first end and a second end substantially opposing the first end, wherein the first end is connected to the first port, and wherein the second end is connected to an input port of a dialyzer;
an arterial blood pump in communication with the machine arterial line and configured to pump fluid from the first end to the second end;
a machine venous line comprising a first end and a second end substantially opposing the first end, wherein the first end is connected to an output port of the dialyzer, and wherein the second end is connected to the second port; and
a substitution fluid line comprising a first end and a second end substantially opposing the first end, wherein the first end is connected to a substitution fluid source, and wherein the second end is connected to the substitution inlet port; and
wherein the first portion of the adjustable valve is movable relative to the second portion to selectively connect the first port and the second port with a selected combination of two of the arterial inlet port, the venous outlet port, or the substitution inlet port to adjust a flow of fluid through the adjustable valve.

8. The machine of claim 7, comprising a control system in communication with the arterial blood pump and configured to control the flow of fluid through the adjustable valve to the machine arterial line.

9. The machine of claim 7, comprising an actuator connected to the adjustable valve and configured to adjust a position of the first portion relative to the second portion in response to a control signal.

10. A method for blood return in a hemodialysis machine using an adjustable valve comprising a first portion having a first port connected to a machine arterial line and a second port connected to a machine veinous line and a second portion having an arterial inlet port connected to a patient arterial access line, a venous outlet port connected to a patient venous access line, and a substitution inlet port connected to a substitution fluid line, the method comprising:
moving the first portion of the adjustable valve to a first position relative to the second portion in which the first port and the second port are connected with a first combination of two of the arterial inlet port, the venous outlet port, or the substitution inlet port;
operating an arterial blood pump in communication with the machine arterial line to control a first flow of fluid through the adjustable valve to the machine arterial line;
moving the first portion to a second position relative to the second portion in which the first port and the second port are connected with a second combination of two of the arterial inlet port, the venous outlet port, or the substitution inlet port; and
operating the arterial blood pump to control a second flow of fluid through the adjustable valve to the machine arterial line.

11. The method of claim 10, wherein moving the first portion relative to the second portion comprises rotating the first portion relative to the second portion.

12. The method of claim 10, wherein moving the first portion relative to the second portion comprises sending a control signal to an actuator connected to the adjustable valve, wherein the actuator is configured to adjust a position of the first portion relative to the second portion in response to the control signal.

13. The method of claim 10, wherein moving the first portion to a first position comprises moving the first portion relative to the second portion such that the first port is in communication with the arterial inlet port and the second port is in communication with the venous outlet port.

14. The method of claim 13, wherein moving the first portion to a second position comprises moving the first portion relative to the second portion such that the first port is in communication with the substitution inlet port and the second port is in communication with the venous outlet port.

15. The method of claim 13, comprising:
moving the first portion to a third position relative to the second portion in which the first port and the second port are connected with a third combination of two of the arterial inlet port, the venous outlet port, or the substitution inlet port; and
operating the arterial blood pump to control a third flow of fluid through the adjustable valve to the machine arterial line.

16. The method of claim 15, wherein moving the first portion to a third position comprises moving the first portion relative to the second portion such that the first port is connected with the substitution inlet port and the second port is connected with the arterial inlet port.

17. The method of claim 15, comprising:
moving the first portion to a fourth position relative to the second portion in which the first port and the second port are connected with a fourth combination of two of the arterial inlet port, the venous outlet port, or the substitution inlet port; and
operating the arterial blood pump to control a fourth flow of fluid through the adjustable valve to the machine arterial line.

18. The method of claim 17, wherein moving the first portion to a fourth position comprises moving the first portion relative to the second portion such that the first port is connected with the venous outlet port and the second port is connected with the arterial inlet port.
